(19) United States
(12) Patent Application Publication
(10) Pub. No.: US 2010/0198140 A1
(43) Pub. Date: Aug. 5, 2010

(54) Percutaneous tools and bone pellets for vertebral body reconstruction

(76) Inventor: Kevin Jon Lawson, Sault Ste. Marie, MI (US)

Correspondence Address: Robert Charles Hill, 235 Montgomery Street #821, San Francisco, CA, 94104, US

(21) Appl. No.: 12/322,637

(22) Filed: Feb. 5, 2009

(51) Int. Cl. A61M 31/00; A61M 25/00

(52) U.S. Cl. 604/57; 604/264

(57) ABSTRACT

A percutaneous surgical tool comprises a cannula with an open slot at the distal end and a closed tip. A variety of articulated and solid tamps with different tip geometries are used to push bone aside to open up a void for filling. Bone pellets are rammed down the hollow interior, lumen, of the cannula by a tamper. A ramp inside the closed end causes the bone pellets to eject out to the side into a void to-be-filled. Variations in the shapes of the pellets and the ends of the tampers vary the orientations of the pellets as they are ejected through the end slot out from the cannula. One tamper with a sharp flat diagonal cut end can be twisted to push the rear end of the pellet harder sideways and out parallel to the cannula. Curved cannulas allow better access to all parts of the void to-be-filled.

### BACKGROUND OF THE INVENTION

1. Field of the Invention

The present invention relates to percutaneous surgical methods and devices to stabilize vertebra, and more particularly to surgical tools and bone pellets for packing voids inside damaged vertebrae.

2. Description of Related Art

Vertebral compression fractures (VCF's) secondary to osteoporosis can occur spontaneously or result from even minor trauma. When the thick block of bone at the front of the vertebra in the spine collapses, the spine can shorten and fall forward. The posterior muscles and ligaments try to counterbalance the bending, making the osteoporotic anterior spine subjected to even larger compressive stresses. Healing of untreated fractures in the deformed state can make the less than optimum biomechanics a permanent impediment in the sufferer's life.

Bones and their surrounding structures will heal more rapidly and more normally if the damaged bone structures are reconstructively returned to their original shapes and positions and any voids in the bone filled with bone grafts or other suitable matrix materials.

Conventional treatments for osteoporotic and pathologic vertebral fractures rely on the application of liquid acrylic glass (PMMA). Such treatments are minimally invasive, and introduce the reconstructive materials into fractured vertebra through small incisions using metal cannulated tools. But the liquid PMMA and other structural graft materials are hard to control with traditional methods. The liquid PMMA can leak into the surrounding areas before it hardens in the right places, and that invasion can cause problems later. Inserting solid materials seems preferable because solids are easier to control and do not flow or migrate on their own like liquids can.

A great number of percutaneous tools and procedures have thus been developed to clean out damaged tissues, expand collapsed spaces with balloons and catheters, and to insert replacement materials like bone grafts, artificial disks, and medicines. One particular tool of interest inserts bone pellets into voids inside the vertebrae through a hollow tube or cannula. See, U.S. Pat. No. 7,238,209, issued Jul. 3, 2007, to Hiromi Matsuzaki, et al.

Different shaped bone pellets can be used according to the nature and size of the bone voids to be filled and packed. Bone grafts provide a framework into which the host bone can regenerate and heal. Bone cells weave into and through the porous microstructure of the implant. The implants provide a framework to support new tissues and bone as they grow to reconnect the fractured segments. Bone cells and living cells inside the graft also stimulate growth of surrounding bone and tissue.

Many bone graft extender materials are commercially available for other applications, and some could be put to good use if they could be appropriately and safely placed down within the vertebra. “PRO OSTEON IMPLANT-500” is one such artificial bone graft material, and it is made from marine coral exoskeletons. Its porous structure mimics the porosity of human cancellous bone. PRO OSTEON IMPLANT-500 facilitates the natural healing process without risking disease transmission, biological rejection, and the additional surgery necessary to collect donor bone for grafting.

Such bone void fillers are clinically proven materials that have changed the way orthopedic surgeons do bone grafts. PRO OSTEON IMPLANT-500 is sterile, biocompatible, and can be easily molded to fill a defect in fractured bones. It is approved by the Food and Drug Administration (FDA) when used with rigid internal fixation for metaphyseal fracture defects, e.g., fractures at the ends of the long bones of the arms and legs.

Balloon kyphoplasty inserts a balloon-like device, an inflatable bone tamp, into a channel drilled into a fractured vertebra. The tamp is positioned in the vertebral body and inflated to create a void for filling to restore the normal height of the vertebral body. The KyphX® Exact™ Inflatable Bone Tamp and the KyphX® Elevate™ Inflatable Bone Tamp are directional inflatable bone tamps (IBT's) marketed by Kyphon Inc. (Sunnyvale, Calif.) to provide targeted balloon inflation for fracture reduction and cavity creation during Balloon Kyphoplasty procedures. The KyphX Directional IBTs are compatible with the KyphX Osteo Introducer, KyphX Advanced Osteo Introducer and KyphX One-Step Osteo Introducer Systems. Directional balloons can be used for cavity creation and fracture reduction, depending on fracture morphologies, bone quality, and access channel trajectory.

Closed-tip cannulas are well known. The Katena cannula K7-3016 (Katena Products, Inc, Denville, N.J.) is a 23-gauge cannula that features an end-opening slot for direct irrigation and a tapered tip for ease of entry into an undilated punctum. The 13-mm length makes it ideal to probe as well as irrigate the proximal lacrimal system. Katena cannula K7-3016 eliminates the need for punctal dilation and placement of Bowman probes to dilate the eye's punctum and measure canalicular obstruction, respectively.

### SUMMARY OF THE INVENTION

Briefly, a percutaneous surgical tool embodiment of the present invention comprises a cannula with an open slot at the distal end and a closed tip. A variety of articulated and solid tamps with different tip geometries are used to push bone aside to open up a void for filling. Bone pellets are rammed down the hollow interior, lumen, of the cannula by a tamper. A ramp inside the closed end causes the bone pellets to eject out to the side into a void to-be-filled. Sometimes the pellets are forcefully driven in by pounding on the tamps, much like a pile-driver operates. Variations in the shapes of the pellets and the ends of the tampers vary the orientations of the pellets as they are ejected through the end slot out from the cannula. One tamper with a sharp flat diagonal cut end can be twisted to push the rear end of the pellet harder sideways and out parallel to the cannula. Curved cannulas allow better access to all parts of the void to-be-filled.

The above and still further objects, features, and advantages of the present invention will become apparent upon consideration of the following detailed description of specific embodiments thereof, especially when taken in conjunction with the accompanying drawings.

### BRIEF DESCRIPTION OF THE DRAWINGS

FIG. 1A is a perspective view diagram of a closed-tip cannula, a bone graft pellet, and a flat tipped tamp in an embodiment of the present invention in which the cannula is inserted into the interior of a vertebral body and many bone graft pellets are pushed in by pounding the tamp behind them;

FIGS. 1B-1D are perspective view diagrams of the closed-tip cannula of FIG. 1A and a wedge-tipped tamp that can be inserted, as in FIG. 1C, and used to laterally push aside a bone graft pellet loaded in the slot at the end of the cannula by twisting the wedge tip, as in FIG. 1C;

FIGS. 2A and 2B are top and side, partial cross section views of a human vertebra showing how the cannula and tamps of FIGS. 1A-1D would be positioned for use during percutaneous surgery, and several bone graft pellets are shown having already been delivered to the interior of the vertebral body;

FIG. 3 is a flowchart diagram of a method embodiment of the present invention that recites the typical steps involved in the percutaneous surgery of the vertebra shown in FIGS. 2A and 2B, the guide needles and pins are used for open-tip cannulas and are not needed with the closed-tip cannulas of FIGS. 1A-1D, and 2A and 2B;

FIGS. 4A and 4B are side cross section and top plan view diagrams of the tip of a closed-tip cannula embodiment of the present invention;

FIGS. 5A and 5B are side and top view diagrams of the distal end of a blunt nose flexible tamp embodiment of the present invention with leaf joints that can be used with the closed-tip cannula of FIGS. 1A-1D, 2A-2B, 3, and 4A-4B, to pound bone graft pellets into the interior spaces of the vertebral body of FIGS. 2A and 2B;

FIGS. 6A and 6B are side and top view diagrams of the distal end of a blunt nose articulated tamp embodiment of the present invention with a single spring linked joint that can be used with the closed-tip cannula of FIGS. 1A-1D, 2A-2B, 3, and 4A-4B, to pound bone graft pellets into the interior spaces of the vertebral body of FIGS. 2A and 2B;

FIGS. 7A and 7B are side and top view diagrams of the distal end of a blunt nose articulated tamp embodiment of the present invention with three linked joint that can be used with the closed-tip cannula of FIGS. 1A-1D, 2A-2B, 3, and 4A-4B, to pound bone graft pellets into the interior spaces of the vertebral body of FIGS. 2A and 2B;

FIG. 8 is a side view diagram of a variety of bone graft pellets useful in various embodiments of the present invention;

FIG. 9 is an enlarged perspective view diagram of the distal end of a closed-tip cannula embodiment of the present invention as shown in FIGS. 1A-1D, 2A-2B, 3, and 4A-4B;

FIGS. 10A-10C are cutaway side view diagrams of the distal end of a closed-tip cannula embodiment of the present invention showing how a tamp like that of FIGS. 5A-5B articulates on its leaf joints as it is pushed forward, and showing how it can be directed to push bone graft pellets and soft interior bone sideways while within a vertebral body;

FIG. 11A is a cutaway side view diagrams of the distal end of a closed-tip cannula embodiment of the present invention showing how a tamp like that of FIGS. 7A-7B articulates on its three link joints as it is pushed forward, and showing how it can be directed and pounded to push bone graft pellets and soft interior bone sideways while within a vertebral body; and

FIG. 11B is a cutaway side view diagrams of the distal end of an open-tip cannula with an oblique end, in another embodiment of the present invention; and

FIGS. 12A-12D are a sequence of diagrams showing how an orderly build up of bone graft sections inside the void of a vertebral body during percutaneous surgery can be assisted by the oblique faces of the grafts and tools used.

### DETAILED DESCRIPTION OF THE INVENTION

Percutaneous access to a vertebral body is an established and medically accepted procedure for treating a variety of conditions. Kyphon brand balloon tamps are probably the most widely used instruments. An alternative is vertebroplasty, in which simple injections of liquid or paste bone cements are pumped down a large caliber needle into the cancelous part of weakened or fractured vertebrae. The most common bone cement is probably polymethylmethacrylate (PMMA).

In embodiments of the present invention, commercially available solid pellets of substitute bone are placed as grafts into to the cancelous parts of weakened or fractured vertebrae with cannulas impaction tools. A variety of lengths and shapes are selected that will best fill the voids using impaction grafting. Filling the voids this way can also re-expand and restore the vertebral body to a more normal configuration.

The key to success is to use both the appropriate impaction tools and graft bone pellets with the optimum sizes, lengths, diameters, and mechanical properties. Simple autogenous or allograft bone would not suffice. Using containment meshes has also proven to be too costly and difficult for wide acceptance.

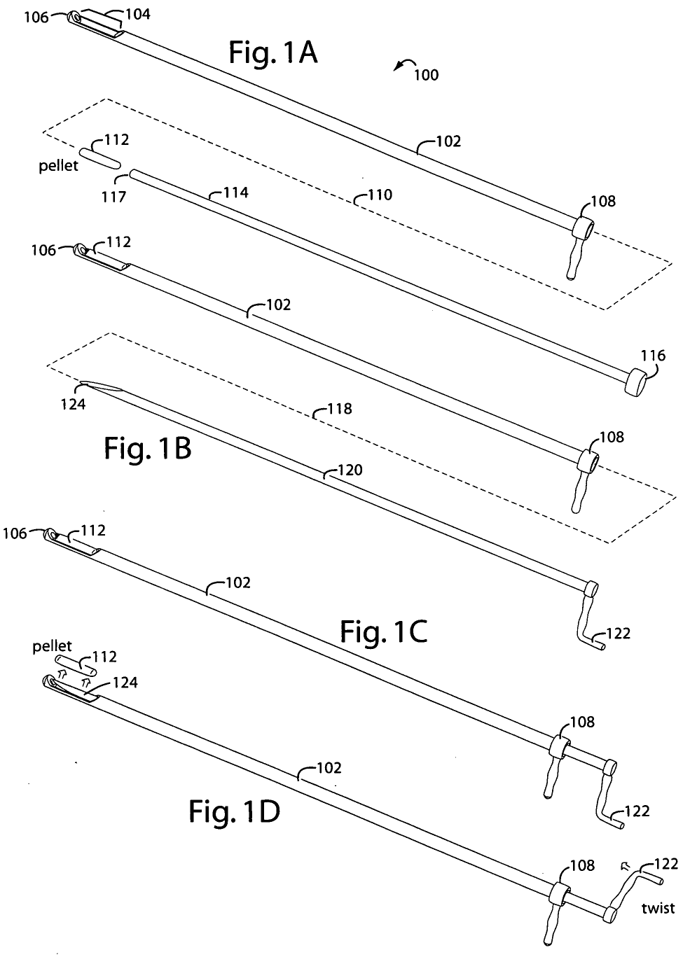

FIGS. 1A-1D represents closed-tip cannulas, bone graft pellets, and tamps included in a system embodiment of the present invention, and are referred to herein by the general reference numeral 100. System 100 includes a cannula 102 with a side slot 104 and closed tip 106 on its distal end. A handle 108 provides some leverage to twist the cannula 102 to best position side slot 104. Cannula 102 is typically inserted into the interior of a vertebral body during percutaneous surgery. A loading 110 of a bone graft pellet 112 is followed by a tamp 114 with an anvil 116 and a flat nose 117. Many bone graft pellets 112 of various sizes and shapes can be pushed into the interior of a vertebral body by ramming the tamp 114 behind them.

FIGS. 1B-1D show how loading 118 a tamp 120 with a handle 122 and a wedge tip 124 can be used after bone graft pellet 112 is readied. Twisting handle 122 will laterally eject bone graft pellet 112 from slot 104, as in FIG. 1D. The action is similar to the ejecting of a spent cartridge from the slot of a rifle.

Handles 108 and 122 also serve as stops to prevent over-penetration of the tools into the surgical site.

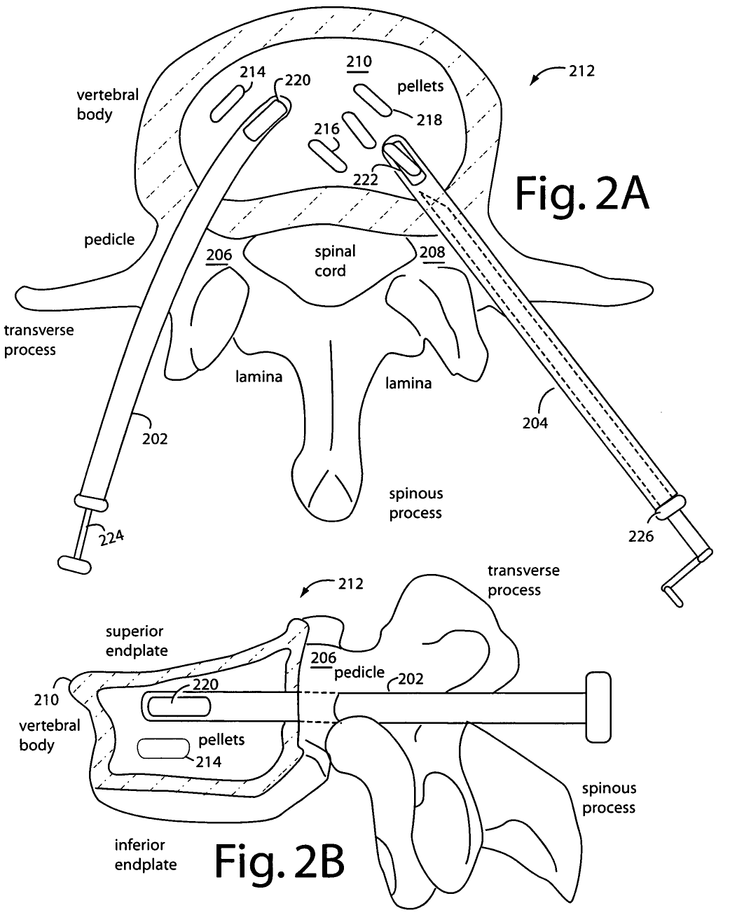

FIGS. 2A and 2B illustrate a method in which a cannula 202 from the left and a cannula 204 from the right are inserted through holes drilled through pedicles 206 and 208 into the vertebral body 210 of a vertebra 212 for use during percutaneous surgery. As an example, cannula 202 is shown as a curved type. A straight one could also be used. Several bone graft pellets 214, 216, and 218 are shown already having already been delivered to the interior of the vertebral body through slots 220 and 222 in cannulas 202 and 204. Here, a tamp 224 has been used to ram down the pellets through the cannulas. A wedge-tipped rod 226 could also be inserted and twisted to expel each pellet.

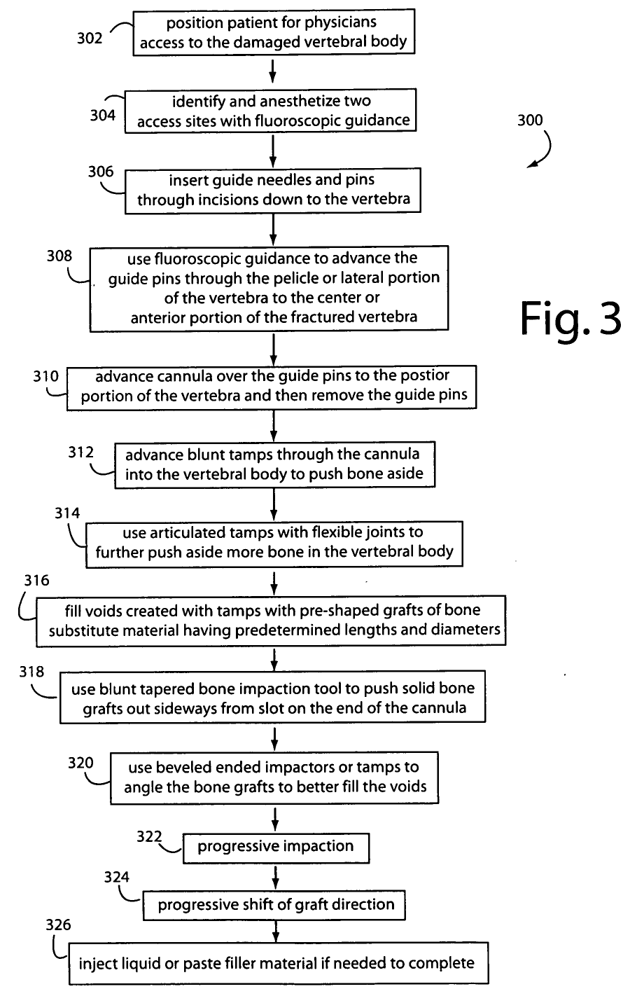

FIG. 3 represents a percutaneous bone graft method embodiment of the present invention, and is referred to herein by the general reference numeral 300. In a step 302, the patient is positioned for access to a damaged vertebral body. In a step 304, two access sites are identified with fluoroscopic guidance and anesthetized. If open-tipped cannulas are being used, guide needles and pins are inserted through incisions down to the vertebra in a step 306. Fluoroscopic guidance is used in a step 308 to advance the guide pins through a pedicle or lateral portion of the vertebra to the center or anterior portion of a fractured vertebra. In a step 310, a cannula is advanced over the guide pins to the posterior portion of the vertebra. Then the guide pins can be removed.

In a step 312, blunt tamps are pushed through the cannula into the vertebral body to force soft bone aside. In a step 314, tamps with flexible joints are used to further push aside more bone inside the vertebral body. A step 316 fills the voids created by the tamps with pre-shaped grafts of bone substitute material having predetermined lengths and diameters. In a step 318, blunt-tapered bone impaction tools are used to push solid bone grafts out sideways from a slot on the end of a closed-tip cannula. In a step 320, beveled ended impactors or tamps are used to angle the bone grafts to better fill the voids. A step 322 uses progressive impaction. A step 324 includes progressively shifting the graft direction. A step 326 injects liquid or paste filler material if needed to complete the procedure.

In another embodiment of the present invention, access is made to the vertebral body through standard percutaneous fluoroscopically guided techniques with needles and hollow cannulae. Bone grafts and augment devices are impacted with cannulated tools with a circular impactor. Various nose shapes on the impaction tools provide for lateral displacement. For example, oblique flat faces on the noses and tails of the bone grafts and tools help stack the pieces side by side inside the voids.

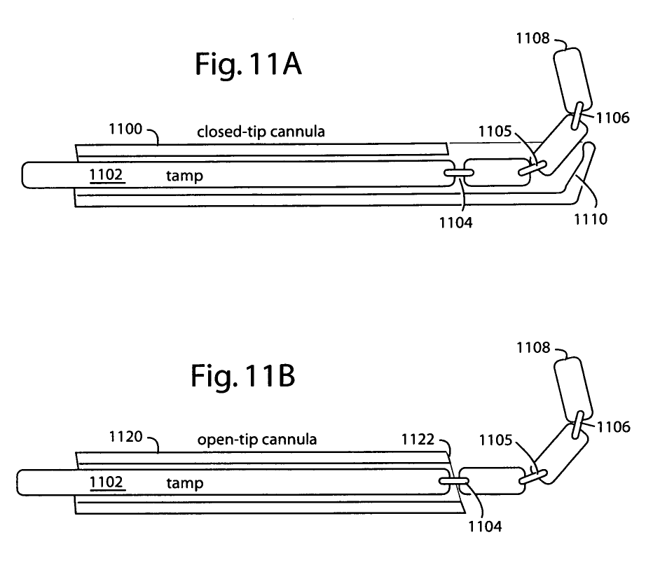

Referring to FIG. 3, a similar method of percutaneous surgical repair of a damaged vertebral body comprises placing a cannula or dilating obturator and then a cylindrical cannula over a guide pin. The cannula may have an oblique side, as in FIG. 11A, to allow translation of grafts in controlled directions. Each graft is placed by impaction with a tamp. A tapered oblique tool can be used to push or tap behind the graft using a mallet. The grafts can be directed to one side by virtue of the oblique end on the cannula. The tamp is rotated periodically to help fill grafts in all around, advancing a full cylinder tamp or spring tool to push each graft section in further. The progressive build up will combine to support a fractured vertebra with grafts to help expand and reshape a crushed structure.

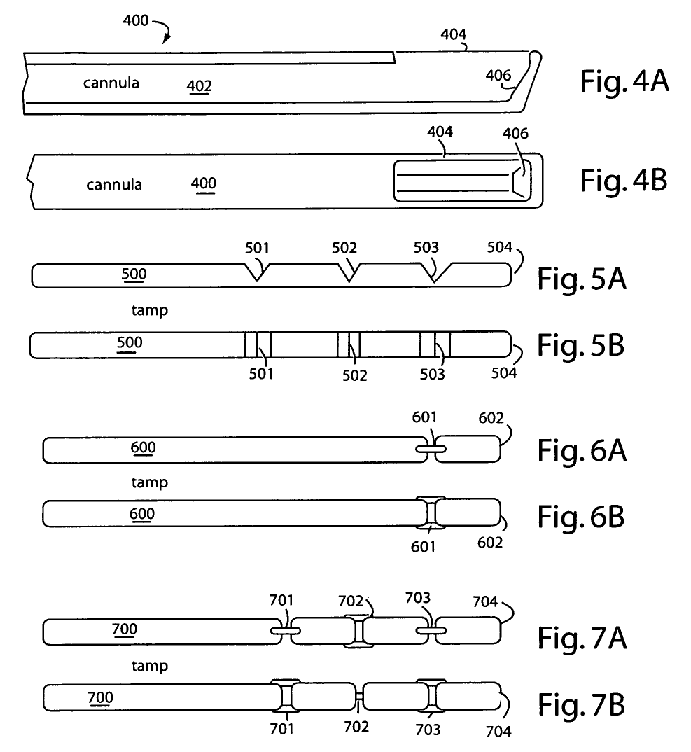

FIGS. 4A and 4B represent a closed-tip cannula embodiment of the present invention, referred to herein by the general reference numeral 400. Cannula 400 includes a hollow interior lumen 402 that terminates at the distal end with a side slot 404 in the shape of a slot. A ramp 406 helps materials pushing down inside lumen 402 to be redirected out to the side from side slot 404. Cannula 400 can be straight or curved, e.g., to allow better access to portions of the interior of a vertebral body through a single incision.

FIGS. 5A and 5B represent the distal end of a blunt nose flexible tamp embodiment of the present invention, referred to herein by the general reference numeral 500. Tamp 500 has one or more leaf joints 501-503 that can be used with a closed-tip cannula to pound bone graft pellets into the interior spaces of a vertebral body, such as in FIGS. 2A and 2B. A nose 504 can have a variety of useful shapes. FIGS. 5A and 5B show a blunt nose, but pointed, rounded, concave, and wedge shaped noses all have important applications. Tamp 500 is made of metals or plastics that are strong enough to survive being pounded, and that are biocompatible.

FIGS. 6A and 6B represent the distal end of a blunt nose flexible tamp embodiment of the present invention, referred to herein by the general reference numeral 600. Tamp 600 has one or more link joints 601 that can be used with a closed-tip cannula like cannula 400 in FIGS. 4A and 4B to pound bone graft pellets into the interior spaces of a vertebral body as in FIGS. 2A and 2B. The distal end can thus flex in two opposite directions. A nose 602 can have a variety of useful shapes. FIGS. 6A and 6B show a blunt nose, but pointed, rounded, concave, and wedge shaped noses all have important applications. Tamp 600 is made of metals or plastics that are strong enough to survive being pounded, and that are biocompatible.

FIGS. 7A and 7B represent the distal end of a multi-link blunt nose flexible tamp embodiment of the present invention, referred to herein by the general reference numeral 700. Tamp 700 has two or more link joints 701-703 that can be used with a closed-tip cannula like cannula 400 in FIGS. 4A and 4B to pound bone graft pellets into the interior spaces of a vertebral body as in FIGS. 2A and 2B. Here, links 701 are orthogonal in action to links 702, permitting flexing of the distal end in two orthogonal directions. A nose 704 can have a variety of useful shapes. FIGS. 7A and 7B show a blunt nose, but pointed, rounded, concave, and wedge shaped noses all have important applications. Tamp 700 is made of metals or plastics that are strong enough to survive being pounded, and that are biocompatible.

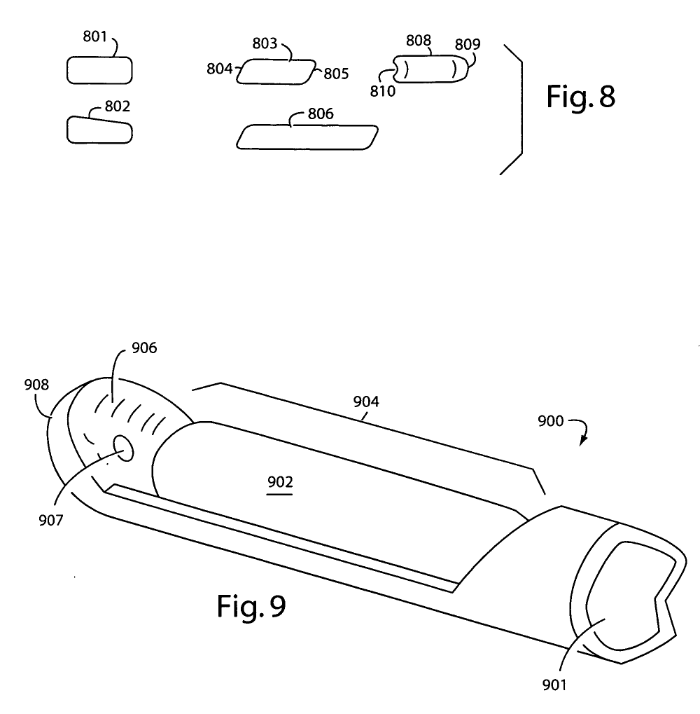

FIG. 8 is a side view diagram of a variety of bone graft pellets useful in various embodiments of the present invention. For example, a pellet 801 is made of a solid material similar to “PRO OSTEON IMPLANT-500”, and has a simple cylindrical shape sized to slide down inside lumen 402 of cannula 400 and slot sideways out of 404 (FIGS. 4A and 4B). A pellet 802 is a flat faced round wedge, and a pellet 803 is a solid cylinder with oblique opposite faces 804 and 805. A pellet 806 is similar but longer in length. A pellet 808 is bullet shaped with a convex nose 809 and a concave tail 810. The various shapes and lengths can interlock and help self-assemble a mass of these pellets into a framework within a void in a vertebral body.

FIG. 9 represents the distal end 900 of a closed-tip cannula embodiment of the present invention, such as in FIGS. 1A-1D, 2A-2B, 3, and 4A-4B. A hollow cylinder 901 runs the full length and allows guide wires, tools, and bone grafts to be passed through. A bone graft pellet 902 is shown ready to be ejected from a slot 904 in the side. An inclined ramp 906 is situated to help with the sideways ejection of pellet 902. A small concentric hole 907 through a closed tip 908 is provided for guide wires that help with the initial positioning of cannula 900. A typical diameter for hole 907 is 0.7-1.0 millimeters in a tip 908 that is 4.5-5.0 millimeters in diameter. Closed tip 908 is shaped to make insertion into a small incision simple and easy by having a blunt tip that pushes tissues aside as it penetrates. Cannula 900 is made of metals or plastics that are strong enough to survive being pounded and twisted against bone, and that are biocompatible. For example, stainless steel. A material is biocompatible if it allows the body to function without allergic reactions, complications, or other adverse side effects.

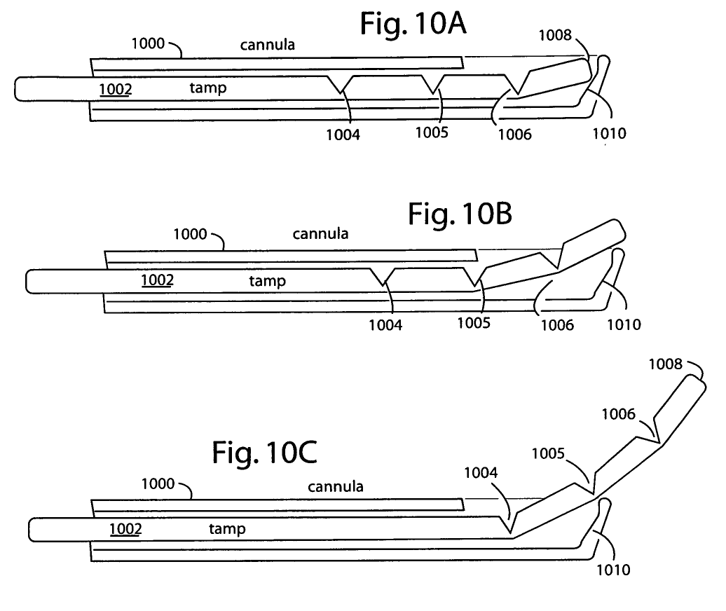

FIGS. 10A-10C represent the distal end of a closed-tip cannula 1000 and a tamp 1002, like those of FIGS. 4A-4B and 5A-5B. Tamp 1002 articulates on its leaf joints 1004-1006 as it is pushed forward. Its nose 1008 slides up a ramp 1010 and out, as shown in FIGS. 10B and 10C. The tamp 1002 can be directed to push bone graft pellets and soft interior bone sideways while within a vertebral body. How far the tamps can be pushed through the cannulas is limited.

FIG. 11A represents the distal end of another closed-tip cannula 1100 and a tamp 1102, like that of FIGS. 7A-7B, articulates on its three link joints 1104-1106 as it is pushed forward. It too can be directed and pounded to push bone graft pellets and soft interior bone sideways while within a vertebral body. Its nose 1108 slides up a ramp 1110 and out.

Variety in the lengths, shapes, and diameters of the bone graft solids are important to the practical application of embodiments of the present invention. Extrusions of plasticized replacement bone matrix could also be forced down large diameter cannulas in sectional lengths using tamps as pistons. Bone tamps with articulated ends and noses with different shapes help make the job of creating a suitable void less difficult and produce better results. The materials used in these tamps are bio-safe metals and plastics, so as not to pose a danger if pieces are inadvertently or accidently left behind.

If any injectable liquid or paste bone cements are used to finish up, the volume of solid bone pellet material impacted into the voids very much reduces or eliminates how much bone cement will really be needed to complete the procedure. Thus safety is inherently improved.

FIG. 11B represents the distal end of an open-tip cannula 1120 with an oblique end 1122, in another embodiment of the present invention. Tamp 1102 articulates on its three link joints 1104-1106 as it is pushed forward. It can be directed and pounded to push bone graft pellets and soft interior bone while within a vertebral body.

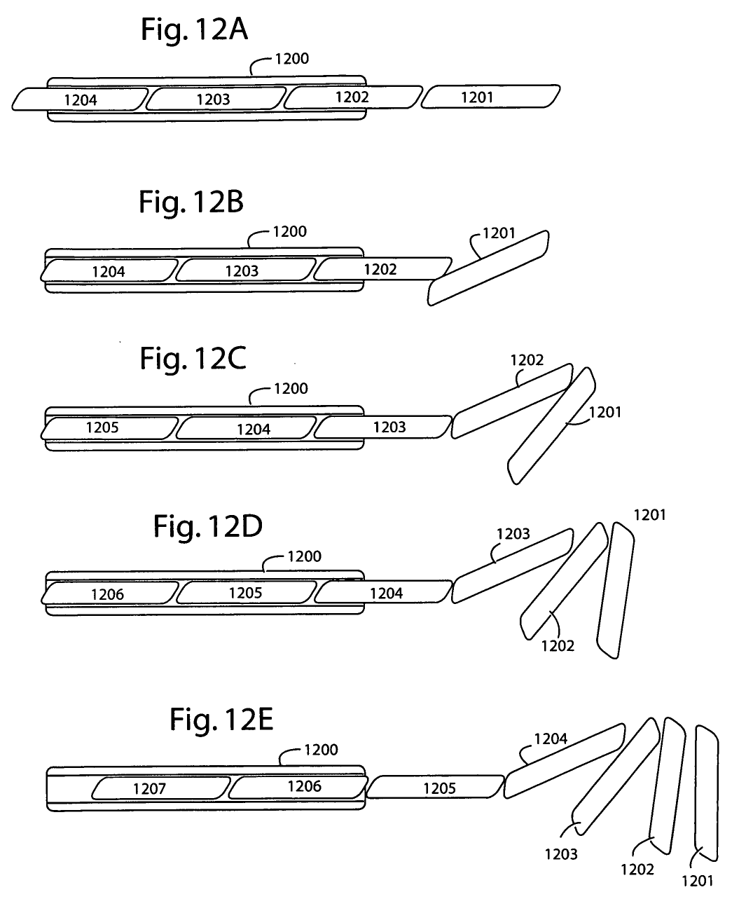

FIGS. 12A-12D show an open-tip cannula 1200 in situ during use and how it can be used to deliver stacks of bone graft sections 1201-1207. Each has an oblique, tilted face that will kick-off to one side when each bone graft section 1201-1207 exits the end of cannula 1200. A tamp 120 like that illustrated in FIG. 1B could be used to control which radial direction the bone graft sections 1201-1207 build up. Differently faced bone graft sections 1201-1207 and tamps will produce other kinds of stacking actions. Tamps 400, 500, 600, and 700 shown in FIGS. 4A, 4B, 5A, 5B, 6A, 6B, 7A, and 7B, could be used effectively as well.

In one tools technique sequence, a cannula or dilating obdurate and then a cylindrical cannula is placed over a guide pin. The cannula may have an oblique side to allow translation of grafts in controlled directions. Each graft is placed or impacted by pounding. A tapered oblique tool is pushed or tapped in behind with a mallet. After partially translating the graft, the tamp is rotated to translate the section further. A full cylinder translating tamp or spring tool is advanced to push the graft sections in further. For example, tools 500, 600, and 700, with wedge or conical point noses. A second device is placed and moved side to side and up and down to progressively build up and support the fractured vertebra. Such can also expand and reshape a crushed structure.

The solid bone grafts of the present invention can further be round, hexagonal, or octagonal in lateral cross section.

Although particular embodiments of the present invention have been described and illustrated related to vertebrae, such is not intended to limit the invention. The treatment of other fractured and weakened bones in the rest of the body is also included. Modifications and changes will no doubt become apparent to those skilled in the art, and it is intended that the invention only be limited by the scope of the appended claims.

The invention claimed is:

1. A percutaneous surgical system, comprising:
a variety of solid bone graft pellets in various shapes and lengths;
a cannula with an interior lumen having a large enough inside diameter to pass any of said variety of solid bone graft pellets;
a side slot disposed in a distal end of the cannula and configured to allow said variety of solid bone graft pellets to be ejected out; and
a tamp sized to fit the cannula and providing a mechanism to force any of said variety of solid bone graft pellets down through the cannula and out the side slot.

2. The percutaneous surgical system of claim 1, further comprising:
a closed tip disposed on the end of the cannula and in front of the side slot, and providing for percutaneous entry into the vertebral body of a vertebrae.

3. The percutaneous surgical system of claim 1, wherein the cannula and tamp are curved along their lengths to allow increased access to the interior of said vertebral body through an incision.

4. The percutaneous surgical system of claim 1, wherein the variety of solid bone graft pellets are comprised of exoskeletons of marine coral.

5. The percutaneous surgical system of claim 1, wherein the variety of solid bone graft pellets include cylindrical shapes with blunt, bullet, pointed, wedge, and oblique ends.

6. The percutaneous surgical system of claim 1, wherein the variety of solid bone graft pellets include various lengths.

7. The percutaneous surgical system of claim 1, further comprising:
an articulated tamp with a distal end that can flex in one direction after being introduced through the cannula into the interior of said vertebral body.

8. The percutaneous surgical system of claim 1, further comprising:
an articulated tamp with a distal end that can flex in two opposite directions after being introduced through the cannula into the interior of said vertebral body.

9. The percutaneous surgical system of claim 1, further comprising:
an articulated tamp with a distal end that can flex in orthogonal directions after being introduced through the cannula into the interior of said vertebral body.

10. The percutaneous surgical system of claim 1, further comprising:
bone cement for injection through the cannula into the interior of said vertebral body to fix said pellets together.

11. A method of percutaneous surgical repair of a damaged vertebral body, comprising:
placing a cannula or dilating obturator and then a cylindrical cannula over a guide pin, wherein said cannula may have an oblique side to allow translation of grafts in controlled directions;
placing each graft by impaction with a tamp;
using a tapered oblique tool to push or tap behind said graft;
partially translating a graft, then rotating the tamp to translate each section further;
advancing a full cylinder translating tamp or spring tool to push each graft section in further; and
progressively building up and support a fractured vertebra with said grafts to expand and reshape a crushed structure.

12. The method of claim 11, further comprising:
using solid bone grafts that are round, hexagonal, or octagonal in lateral cross section.
